(19) United States
(12) Patent Application Publication
(10) Pub. No.: US 2008/0103332 A1
(43) Pub. Date: May 1, 2008

(54) METHOD OF MANUFACTURING PTA OXIDIZED CATALYST WITH LOWER CORROSION

(76) Inventor: David YEN, Tao-Yuan Hsien (TW)

Correspondence Address: SINORICA, LLC, 528 FALLSGROVE DRIVE, ROCKVILLE, MD, 20850, US

(21) Appl. No.: 11/555,618

(22) Filed: Nov. 1, 2006

(51) Int. Cl. C07C 51/255; B01J 27/132

(52) U.S. Cl. 562/412; 502/228

(57) ABSTRACT

A method of manufacturing PTA oxidized catalyst with lower corrosion, the oxidized catalyst is brominated cobalt manganese acetate in solution with a weight ratio of Co/Mn/Br being in a predetermined ratio. When the brominated cobalt manganese acetate in solution meets a condition (Co×2.7)+(Mn×2.9)>Br, CoBr₂, MnBr₂, cobalt acetate and manganese acetate are used as sources for manufacturing; when the brominated cobalt manganese acetate in solution meets the condition (Co×2.7)+(Mn×2.9)<Br, CoBr₂, MnBr₂ and micro amount of bromine are used as sources for manufacturing without adding or with adding therein only with micro amount of HBr, corrosiveness of the oxidized catalyst is lowered.

### BACKGROUND OF THE INVENTION

1. Field of the invention

The present invention relates to a method of manufacturing PTA oxidized catalyst with lower corrosion, and especially to a method of manufacturing oxidized catalyst by CoBr₂ and MnBr₂.

2. Description of the Prior Art

Purified Terephthalic Acid (PTA) is used widely as industrial production raw material; taking polyester in production of clothing as an example, its main material is polyethylene terephthalate (PET), generally, the main way of production in the industry is to make esterifying and polycondensation of PTA and ethylene glycol (EG) to obtain PET.

Terephthalic acid is made by reaction of para-xylene (PX) with O₂, by virtue that the atoms in an oxygen molecule are in a state of triplet spin, they will not react with carbon atoms on a hydrocarbon compound (CPX) in a state of single spin to do chemical oxidation, hence during the process of production, there must be complex salts formed of at least a kind of heavy metallic compound containing no acetic acid group (mainly from Co, Mn, Cr, Ni and Cu etc.), transition metals and solvent acetic acids, while these metallic acetic acids make metallic ions able to absorb oxygen molecules by the combining force generated in the coordination complexing field to change their basic states to become the state of single spin easier for reaction, the reaction of oxidation of the para-xylene (PX) after the catalyzed initiating reaction is a kind of spontaneous oxidation, that is, just a kind of initiating reaction of transition metal can generate free bases of sufficient concentration to render the amplitude of PX oxidation spread continuously.

However, after initiating reaction, transition cobalt metal itself is oxidized to become cobalt of three valences, under a higher PX oxidation reacting temperature, it is very easily to proceed to decarboxylating reaction to respectively form carbon dioxide, methyl acetate, benzene m-tricarbonic acid, benzoic acid, phenol and diphenyl etc., this leaves problems of cost and quality of materials.

Cobalt of three valences required in initiating reaction is not asked for too high concentration, but when the accumulated amount of it is overly large, the above mentioned problem is induced; the way to improve is to take advantage of complexing of a kind of stable coordination group with cobalt of three valences to shorten the life period of Co⁺³ in acetic acid to the minimum, and to study for getting a kind of bromide that is easiest to combine with a kind of cobalt salt to meet the subsequent demand, and to take advantage of adopted concentration of acetic acid to control the rate of oxidation and the rate of selection of oxidation (Partenheimer et al. U.S. Pat. No. 5,081,290). Nowadays, it is typical to use the catalysis of the ions of cobalt, manganese, bromine etc. to render rates of reactions and qualities of products to reach their optimal conditions.

PTA oxidized catalysts at an early stage are taken from solid cobalt compounds, solid manganese compounds and liquid bromides respectively added and mixed to get concentrations required for manufacturing PTA.

By the fact that solid acetic acid and acetate manganese are subjected to be condensed into lumps because of dehydration, they are extremely hard to be stored, and PTA manufacturers must consume manpower and working hours to resolve the solid cobalt, manganese compounds and liquid bromic acid in manufacturing, this is very inconvenient, thereby acetate cobalt solution and acetate manganese solution were gradually used to blend with each other to form cobalt manganese acetate in solution (CMA) for use by the PTA manufacturers, so that the manufacturers can save the operations and cost of crystallizing, separating, drying and packing etc.

Each PTA manufacturer demands his own ratio of weight of cobalt, manganese and bromide according to the requirement of production; in using such cobalt manganese acetate in solution (CMA), the PTA manufacturer needs only to add appropriate amount of HBr in pursuance of the requirement of production, in order to supplement bromine required for the catalysis.

While in addition to that HBr is a kind of strong acid, it contains free bromine molecules that is highly dangerous, no matter a PTA manufacturer stores or uses it, he tends to have trouble in relation to environmental conservation and industrial security. And addition of HBr needs manpower. In this view, further HBr is added in cobalt manganese acetate in solution (CMA) to form the recently widely used brominated cobalt manganese acetate in solution (CMB).

However, most Br used in the process of manufacturing of PTA oxidized catalyst is taken from HBr containing quite an amount of free acid and free bromine. No matter in storing or using, the metallic equipment of a PTA manufacturer will be subjected to very large negative affects, including corrosion of the metallic equipment, and even more serious, events of environmental conservation and industrial security may occur.

In related prior art, for example, U.S. Pat. No. 4,051,178 is only disclosed for catalysts the appropriate ratio of Co/Mn/Br and recommended sources of catalysts in manufacturing terephthalic acid as shown below:

Co: (such as cobalt acetate, cobalt naphthenate, cobalt carbonate, cobalt bromide etc.), the recommended concentrations are:

[Co]=200˜600 ppm; preferably 250˜500 ppm; most preferably 300˜400 ppm;

Mn: (manganese acetate, manganese naphthenate, manganese carbonate, manganese bromide etc.), the recommended concentrations are:

[Mn]=0.5˜1.5 times of [Co]; preferably 0.7˜1.3 times of [Co];

Br: (HBr, MnBr₂, CoBr₂, NaBr, KBr, NH₄Br etc.), the recommended concentrations are:

[Br]=more than 2 times of [Co]; preferably 2.5 times of [Co].

Further in prior art, U.S. Pat. No. 4,873,361, for catalysts, only cobalt acetate, manganese acetate plus Br are adopted for adjusting.

In U.S. Pat. No. 6,194,607, for catalysts, it is asserted that third metallic ions such as alkali metal (Li, Na, K, Rb, Cs, Fr) or alkaline earth (Be, Mg, Ca, Sr, Ba, Ra) etc. are added into the presently available Co/Mn/Br catalysts to obtain better reaction performance.

The above stated three patents do not disclose how the Co/Mn/Br catalysts are manufactured to lower their corrosion. The feature of the present invention is a method of manufacturing oxidized catalyst solution with low free acid.

### SUMMARY OF THE INVENTION

In order to solve the trouble of using HBr in the prior art, the inventor of the present invention tried to find a preparation that surely can have least corrosion with different Br sources during the process of study and development; and after using and testing MnBr₂ in lieu of HBr, MnBr₂ is assured to be able to lower corrosion; MnBr₂ also provides a source of manganease. However, if the ratio of Mn/Br in the prescription of a kind of catalyst is lower than 1/2.9, HBr must be added for supplementing for inadequacy. This can rather largely increase the corrosion of CMB. Hence CoBr₂ is added to supplement for inadequacy of bromine.

The object of the present invention is to provide a method of manufacturing PTA oxidized catalyst with lower corrosion; the method can effectively lower corrosion of the oxidized catalyst—brominated cobalt manganese acetate in solution (CMB) to increase operation security in storing and qualities of products in factories.

To obtain the above object, the present invention is provided with a method of manufacturing PTA oxidized catalyst with lower corrosion. When the weight ratio of Co/Mn/Br in a brominated cobalt manganese acetate in solution meets the condition (Co'2.7)+(Mn×2.9)>Br, CoBr₂, MnBr₂, cobalt acetate and manganese acetate are used as sources for manufacturing. When the weight ratio of Co/Mn/Br in a brominated cobalt manganese acetate in solution meets the condition (Co×2.7)+(Mn×2.9)<Br, CoBr₂, MnBr₂ and micro amount of bromine are used as sources for manufacturing without adding or with adding therein only with micro amount of high corrosive HBr.

The present invention will be apparent in its technical content and effect to be achieved after reading the detailed description of the preferred embodiment thereof in reference to the accompanying drawings.

### BRIEF DESCRIPTION OF THE DRAWINGS

FIG. 1 is a chart showing the results of testing Br containing solution in the present invention;

FIGS. 2 and 3 are drawings showing curves in testing corrosion of sampled Br containing solution in the present invention.

### DETAILED DESCRIPTION OF THE PREFERRED EMBODIMENT

The method of manufacturing of the present invention mainly is calculated on the molecular weights of cobalt, manganese and bromine, thus oxidized catalysts with required weight ratios (expressed as Co:Mn:Br=A:B:C) can be easily manufactured.

Wherein: molecular weight of Br/molecular weight of Co=79.909×2/58.93=2.7

Thereby it can be deduced that in CoBr₂, each unit by weight of Co ion contains 2.7 units by weight of Br ion.

And molecular weight of Br/molecular weight of Mn=79.909×2/54.94=2.9

Thereby it can be deduced that in MnBr₂, each unit by weight of Co ion contains 2.9 units by weight of Br ion.

When (A×2.7)+(B×2.9)>C, it needs only to use CoBr₂, MnBr₂, CoAc₂ and MnAc₂ to manufacture brominated cobalt manganese acetate in solution with the weight ratio of Co/Mn/Br desired by PTA manufacturers.

For example, provided that in a kind of catalyst, Co:Mn:Br=A:B:C=1:2:6, we get 1×2.7+2×2.9=8.5>6; thus 1 portion of CoBr₂ used can get 2.7 portions of Br source, 6−2.7=3.3 more portions of Br source is still wanted, as a result of calculation, 3.3/2.9=1.14 portions of MnBr₂ is wanted, thereby, 2−1.14=0.86 portion of Mn is still wanted, that is, same portion of MnAc₂ should be added.

According to such calculation, the ratio CoBr₂:MnBr₂:MnAc₂=1:1.14:0.86 shall be adopted to manufacture brominated cobalt manganese acetate in solution with the weight ratio of Co/Mn/Br desired (1:2:6) without adding HBr. Thus oxidized catalysts made by this method have lower free acid, this is different from the way of adding HBr conventionally.

And for example, provided that (A×2.7)+(B×2.9)<C, CoBr₂, MnBr₂ and micro amount of bromine are used for manufacturing oxidized catalysts.

For example, provided that in a kind of catalyst, Co:Mn:Br=A:B:C=1:2:9, we get 1×2.7+2×2.9=8.5<9; thus 1 portion of CoBr₂ used can get 2.7 portions of Br source, while 2 portions of can MnBr₂ get 2×2.9=5.8 portions of Br, 9-(2.7+5.8)=0.5 portion of Br is still wanted, that is, 0.5 portion of HBr should be added.

According to calculation, the ratio CoBr₂: MnBr₂: HBr=12:0.5 shall be adopted to manufacture brominated cobalt manganese acetate in solution with the weight ratio of Co/Mn/Br desired (1:2:9) with a micro amount of added HBr. Thus oxidized catalysts made by this method have lower free acid; this is different from the way of directly adding HBr conventionally.

The method of the present invention in manufacturing terephthalic acid using the system of brominated cobalt manganese acetate in solution is practiced by well known technique in the art, such as stated in the above mentioned U.S. Pat. No. 5,081,290. It can be practiced by semi-continuing type or batch type. After the initial material para-xylene (PX) is led into a reactor together with acetic acid solvent and an oxidized catalyst, the contents in the reactor are heated to a preferred temperature of about 150 to 205° C., and then are separated and taken out by centrifugal method or filtering method.

In the above process of manufacturing, organic impurities generated by side-reaction and metals generated by corrosion of equipment gradually accumulate in the mother liquid, and must be discharged to control their concentrations, plus exhausting of the part Br in Br₂ and Ch₃Br gases together with exhausting of reaction waste gas and flowing out of the other part including cobalt and manganese together with powder of raw mix, fresh oxidized catalyst thereby shall be supplemented continuously to keep the concentration required by the reaction. The discharged cobalt and manganese powders can be recovered with a recovering system to make oxidized catalyst.

### Corrosion Test Results

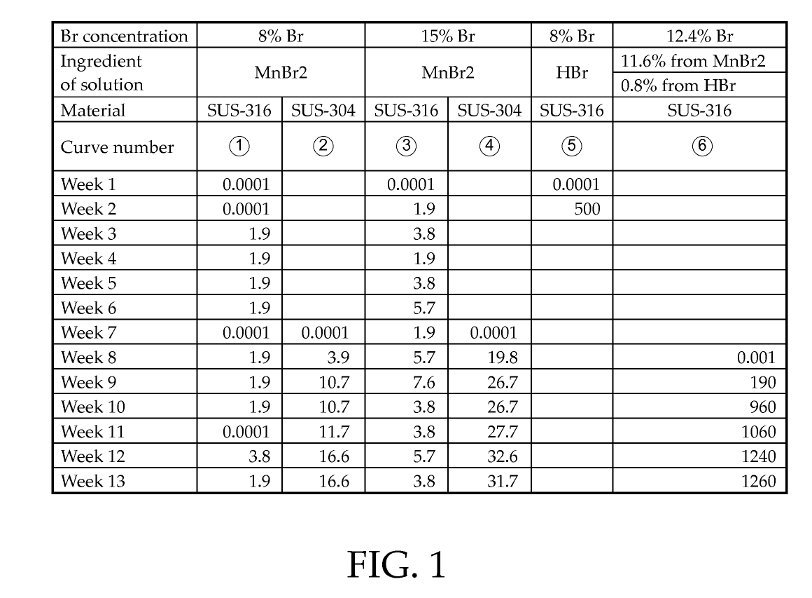

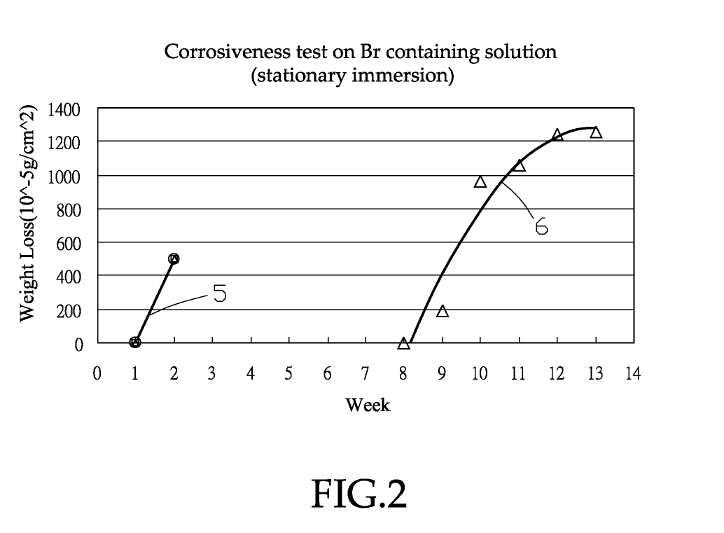

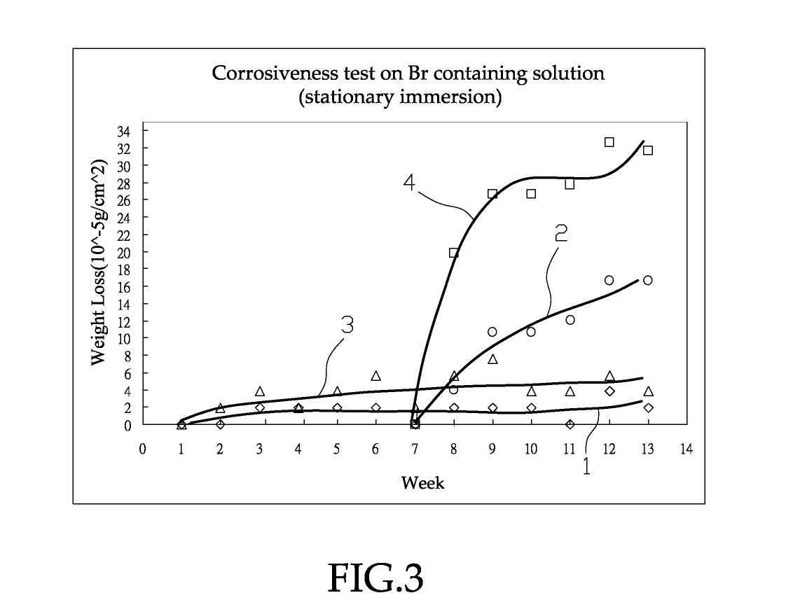

FIG. 1 shows the results of corrosiveness test on Br containing solution in the present invention; FIGS. 2 and 3 are drawings accordingly for contrasting. In the test, there are totally 6 contrast groups, their concentrations of Br are respectively 8%, 15% and 12.4%, the sources of Br are MnBr_{2(aq)} and HBr_{(aq)}. The corrosion testing materials are SUS-316, SUS-304 (these are two kinds of iron sheets with same thickness). The recorded values of the corrosion test are: the reduced weight of each unit area is 10⁻⁵g/cm²; the time unit is: 1 week. The method of the test is stationary immersion.

Referring to FIG. 2, wherein the material for both curves nos.5 and 6 is SUS-316, the ingredient for no.5 is 8% Br (from HBr), for no.6 is 12.4% Br (11.6% Br from MnBr₂; 0.8% Br from HBr).

### Comparison of Corrosion Resisting Time of Steel Sheets

We can see from the drawings that after 8 weeks, the no.6 low free acid curve reacts against the steel sheets, while the no.5 curve being adjusted using HBr for about one week can react instantly; we can see from comparison that, if it is desired to adjust the concentration of Br⁻ ions in the solution, using MnBr₂ for adjusting can get lower corrosion than using HBr, that is, using SUS-316 can get longer corrosion resisting time.

Referring to FIG. 3, in comparison of curves 1˜4 (without adding with HBr) with curves nos.5 and 6 (adding with HBr), we can see:

1. SUS-316 is better than SUS-304 in corrosion resisting.

2. The solution without adding with HBr has lower corrosion resistance against steel sheets.

In comparison of no. 1, 3 with 6, after 13 weeks, lost weights of steel sheets are respectively 1.9, 3.8 and 1260; therefore, adding with HBr can largely increase corrosiveness.

3. We can see from nos. 1˜4 that, corrosiveness of inferior material SUS-304 is not high; this means that corrosiveness of steel sheets is less influential to the concentration of Br ions; while solution free acid (HBr) has extremely large influence to concentration.

In conclusion, as stated above, the method of manufacturing oxidized catalyst provided by the present invention surely can get the effect of lowering corrosiveness.

Having thus described the technical process of my invention, the invention is to be construed as including all modifications and variations falling within the scope of the appended claims.

1. A method of manufacturing PTA oxidized catalyst with lower corrosion, said oxidized catalyst is used in an oxidation process of manufacturing of said PTA (terephthalic acid) and is brominated cobalt manganese acetate in solution with a weight ratio of Co/Mn/Br expressed as A:B:C; said method is:
when said brominated cobalt manganese acetate in solution meets a condition (A×2.7)+(B×2.9)>C, CoBr₂, MnBr₂, cobalt acetate and manganese acetate are used as sources for manufacturing, according to calculation based on values of A, B and C to manufacture said brominated cobalt manganese acetate in solution with said weight ratio of Co:Mn:Br=A:B:C, corrosiveness is lowered.

2. A method of manufacturing PTA oxidized catalyst with lower corrosion, said oxidized catalyst is used in an oxidation process of manufacturing of said PTA (terephthalic acid) and is brominated cobalt manganese acetate in solution with a weight ratio of Co/Mn/Br expressed as A:B:C; said method is:
when said brominated cobalt manganese acetate in solution meets a condition (A×2.7)+(B×2.9)<C, CoBr₂, MnBr₂ and micro amount of bromine are used as sources for manufacturing, according to calculation based on values of A, B and C to manufacture said brominated cobalt manganese acetate in solution with said weight ratio of Co:Mn:Br=A:B:C, corrosiveness is lowered.
